(19) United States
(12) Patent Application Publication
(10) Pub. No.: US 2011/0306796 A1
(43) Pub. Date: Dec. 15, 2011

(54) PROCESS FOR THE PREPARATION OF 1-BROMO-3,5-DIMETHYL ADAMANTANE

(75) Inventors: Ravi Ponnaiah, Vadodara (IN); Ashok Prasad, Vadodara (IN); Piyush Maheshbhai Rana, Vadodara (IN); Kamlesh Shankarlal Kanzariya, Vadodara (IN); Mitulkumar Bharatkumar Dolia, Vadodara (IN)

(73) Assignee: ALEMBIC LIMITED, Gujarat, Vadodara (IN)

(21) Appl. No.: 13/130,953

(22) PCT Filed: Nov. 30, 2009
(86) PCT No.: PCT/IB2009/055407

§ 371 Date: May 24, 2011

(30) Foreign Application Priority Data
Dec. 12, 2008 (IN) 2590/MUM/2008

(51) Int. Cl. C07C 209/08; C07C 17/07

(52) U.S. Cl. 564/459; 570/101

(57) ABSTRACT

The present invention relates to an improved process for the preparation of 1-bromo-3,5-dimethyl adamantane of formula (III), which is an useful intermediate for synthesis of 1-amino-3,5-dimethyl adamantane of formula (I) or pharmaceutically acceptable salt thereof.

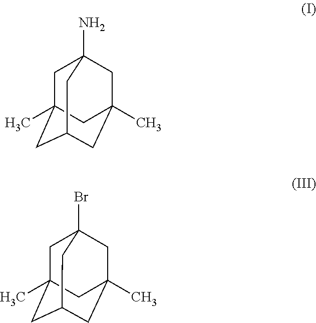

### FIELD OF THE INVENTION

The present invention relates to an improved process for the preparation of 1-bromo-3,5-dimethyl adamatane of formula (III), which is an useful intermediate for synthesis of 1-amino-3,5-dimethyl adamantane of formula (I) or pharmaceutically acceptable salt thereof.

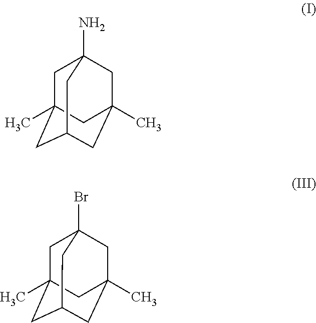

### BACKGROUND OF THE INVENTION

1-amino-3,5-dimethyl adamantane of formula (I) is commonly known as Memantine. Memantine is adamantane derivative. It is an orally active NMDA (N-methyl D-aspartate) receptor antagonist. Memantine is the first in a novel class of Alzheimer's disease medications acting on the glutamatergic system by blocking NMDA glutamate receptors. It is indicated for the treatment of moderate to severe dementia of the Alzheimer's type. Eli Lilly is innovator of Memantine hydrochloride. It is market in US by Forest Labs under the brand name NAMENDA®.

The chemical name of Memantine hydrochloride is 3,5-dimethyl-1-adamantanamine OR 1-amino-3,5-dimethyl adamantane hydrochloride salt. The empirical formula of Memantine hydrochloride is C₁₂H₂₁N.HCl and its molecular weight is 215.76. The CAS Reg. No. of Memantine hydrochloride is [41100-52-1].

Memantine and its hydrochloride salt are covered in U.S. Pat. No. 3,391,142. The process for preparation of Memantine hydrochloride as disclosed in the product patent is as follows:

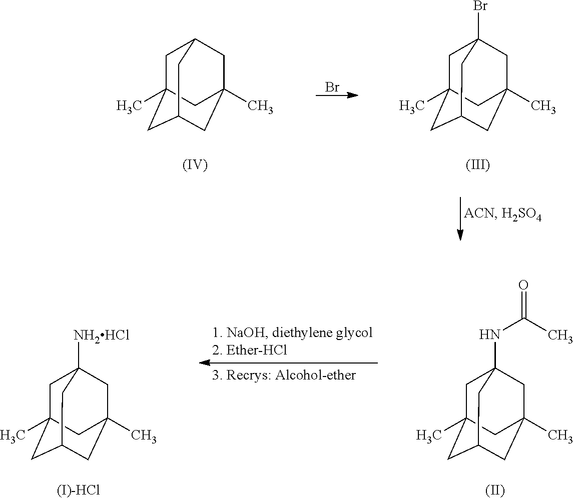

The reaction involves carrying out bromination of 1,3-dimethyl adamantane (IV), using bromine under reflux conditions to obtain 1-bromo-3,5-dimethyl adamantane of formula (III). Compound of formula (III) is further acylated using acetonitrile and sulphuric acid to obtain N-acetamido-3,5-dimethyl adamantane of formula (II) which is further hydrolyzed and converted to Memantine hydrochloride salt (I)-HCl.

WO 2005062724, US 20060173215, WO 2006122238, U.S. Pat. No. 7,405,324, WO 2007096124, WO 2007132476, and several subsequent patents/applications discloses alternative process for the preparation of Memantine hydrochloride.

N-bromo-3,5-dimethyl adamantane of formula (III) is a key starting material for synthesis of Memantine of formula (I). It is a basic requirement that the process for the preparation of KSM should be simple, high yielding, non-hazardous and cost effective.

Bromination reaction is highly exothermic and hazardous when carried out on large scale. Therefore, the scientists of present invention have developed a process for the preparation of compound of formula (III) which is simple, has shorter reaction period, is high yielding, is economically viable and results in improved purity of the product, thereby improving the overall process for the preparation of Memantine (I) or pharmaceutically acceptable salts thereof.

### OBJECT OF THE INVENTION

Therefore, it is a primary object of the present invention is to provide an improved process for the preparation of compound of formula (III).

Another object of the present invention is to provide an improved process for the preparation of compound of formula (I) or pharmaceutically acceptable salts thereof.

### SUMMARY OF THE INVENTION

An aspect of present invention provides an improved process for the preparation of compound of formula (III) comprising of carrying out bromination of compound of formula (IV) using bromine and characterized by carrying out said bromination in the presence of catalytic amount of HBr in acetic acid.

Another aspect of the present invention provides an improved process for the preparation of compound of formula (I) comprising steps of:

(a) carrying out bromination of compound of formula (IV) using bromine and characterized by carrying out said bromination in the presence of catalytic amount of HBr in acetic acid to obtain compound of formula (III)

(b) converting compound of formula (III) in the presence of acetonitrile and sulphuric acid to compound of formula (II)

(c) converting compound of formula (II) to Memantine of formula (I) or pharmaceutically acceptable salts thereof.

Yet another aspect of the present invention provides an improved process for the preparation of compound of formula (I)comprising a step of carrying out bromination of compound of formula (IV) using bromine and characterized by carrying out said bromination in the presence of catalytic amount of HBr in acetic acid to obtain compound of formula (III).

### DETAILED DESCRIPTION OF THE INVENTION

The present invention can be described by synthetic scheme as follows:

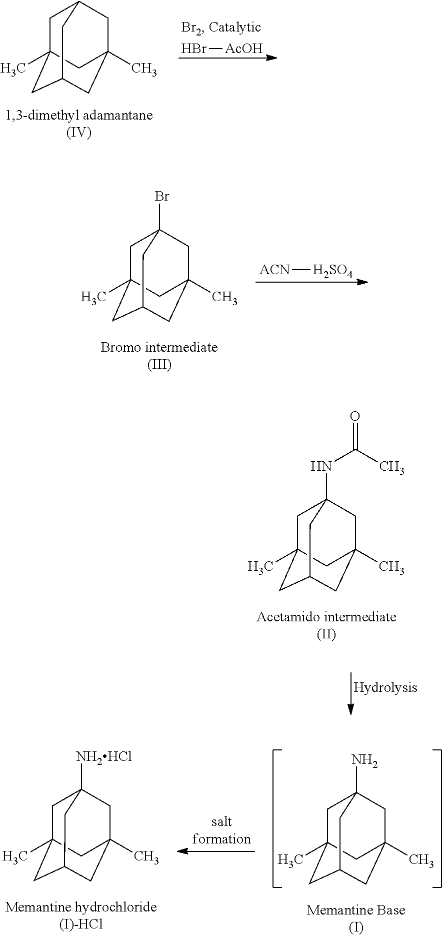

Step (a) comprises of carrying out bromination of 1,3-dimethyl adamantane (IV) characterized by using catalytic amount of HBr in acetic acid. Since bromine itself acts as a medium for the reaction, there is no requirement of using additional solvent. The molar ratio of bromine used is about 4 to 5 molar equivalents with respect to compound of formula (IV). The reaction is usually carried out at about 0° C. to about 55° C., more preferably at about 20° C. to about 50° C.

The addition of bromine is carried out drop-wise in order to control the exothermicity generated during the reaction. Thus, the by-product formation is also controlled. After the reaction is complete the excess unreacted bromine can be recovered by carrying out distillation at atmospheric pressure.

Compound of formula (III) obtained in step (a) can be worked up by common laboratory procedure and used in step (b).

Step (b) comprises converting of compound of formula (III) to compound of formula (II) in the presence of acetonitrile and acetic acid. The said reaction can be carried out preferably in the presence of suitable solvent. Suitable solvent include but is not limited to toluene. The reaction is carried out at about 0° C. to about 60° C. After completion of the reaction, the reaction mass is worked up by routine experimental methods.

Compound of formula (II) obtained in step (b) is converted to compound of formula (I) or pharmaceutically acceptable salt thereof. Hydrolysis is carried out in the presence of base and a suitable solvent. The base comprises of inorganic base including hydroxides, carbonate and bicarbonate of alkali and alkaline earth metal. Examples of base include but are not limited to sodium hydroxide, potassium hydroxide, sodium carbonate, potassium carbonate, sodium bicarbonate, potassium bicarbonate, and the like or the mixtures thereof.

Suitable solvents include but are not limited to 1,2-dimethoxy ethane, 1,4-dimethoxy ethane, polyethylene glycol and the like or mixtures thereof. The reaction is carried out at about 80° C. to about 150° C.

After completion of the reaction the reaction mass may be worked up by general processes known to person skilled in the art. Further compound of formula (I) can be converted to pharmaceutically acceptable salt preferably hydrochloride salt. Alternatively the solvent may be distilled off from the reaction mass and the residue of compound of formula (I) thus obtained can be converted to pharmaceutically acceptable salt preferably hydrochloride salt.

The hydrochloride salt formation may be carried out using hydrochloric acid dissolved in solvents comprising methanol, ethanol, isopropanol, n-propanol, acetone, ethylacetate and the like or mixtures thereof, or by using mixture of concentrated hydrochloric acid in toluene, or by passing hydrochloric gas in solution of compound of formula (I) in solvent comprising methanol, ethanol, isopropanol, n-propanol, toluene, acetone, ethylacetate and the like or mixtures thereof. The product thus obtained is isolated by general processes like centrifugation or filtration.

Salt of compound of formula (I) thus obtained may be crystallized further if desired from solvents comprising methanol, isopropyl alcohol, acetone, toluene, ethylacetate and the like or mixtures thereof.

1. The advantages of present invention are:

2. the reaction time is optimized

3. the molar ration of bromine used for bromination is reduced

4. formation of impurity is reduced

5. the purity of intermediate in increased, thereby improving the quality of final compound.

The following examples illustrate the invention further. It should be understood however, that the invention is not confined to the specific limitations set forth in the individual example but rather to the scope of the appended claims.

### EXAMPLES

### Example—1

### Preparation of 1-bromo-3,5-dimethyl adamantane

Charge 1,3-dimethyl adamantane (100 gm. 0.6 moles) at 25-30° C. Charge HBr in AcOH (1 ml) at 25-30° C. Heat reaction mixture up to 50-55° C. Add drop-wise bromine (124.7 ml, 2.41 moles) slowly at 50-55° C. Maintain it for 12 hours at 50-55° C. Distil out excess bromine atmospherically up to 85° C. Cool down the reaction mixture to 25-30° C. Add MDC (800 ml) into it. Stir for 30 minutes at 25-30° C. Cool MDC reaction mixture to 5° C. Added drop wise previously prepared 1500 ml 5% solution of sodium hydrosulfite in DM Water into reaction mixture. Separate the MDC layer and discard aqueous layer. Wash it twice with DM water (100 ml). Distil out MDC completely atmospherically up to 55° C. Remove the traces of MDC under vacuum (50-100 mm) at 50-65° C. Oily residue is obtained.

Results:—

Dry wt:— 140.0 g

Yield (% w/w):— 1.4

Purity:— 99.7%

### Example—2

### Preparation of N-acetamido-3,5-dimethyl adamantane

Charge 1-bromo-3,5-dimethyl adamantane (100 gm) at 25-30° C. Charge Acetonitrile (100 ml) at 25-30° C. Cool reaction mixture to 5° C. Add conc. H₂SO₄ (200 ml) drop wise at 5-20° C. Raise temperature of reaction mixture to 25° C. Maintain 25-30° C. temperature of reaction mixture for 3 hours. Heat the reaction mixture to 45° C. Maintain reaction mixture at 45-50° C. for 8 hours. Cool reaction mixture to 30° C. Add reaction mixture into ice cold water. Add Toluene to reaction mass. Add sodium hydrosulfite (1 gm). Stir reaction mixture for 15 min. Settle the layers. Separate aqueous layer. Toluene layer is washed with DM water. Settle the layers. Separate aqueous layer. Combine aqueous layers. Stir aqueous layer with toluene. Settle the layers. Separate aqueous layer. Combine toluene layers. Toluene layer is washed with 2% Sodium bi-carbonate solution. Distill out toluene under vacuum till volume of reaction mass remains 200-250 ml up to temp 60° C. Cool reaction mixture to 30° C. Further, cool reaction mixture to 0-5° C. Maintain it at 0-5° C. for 1 hr. Filter the solid at 0-5° C. Wash solid with chilled toluene at 0-5° C. Dry the solid under vacuum at 45-50° C. 2^{nd} crop is isolated from mother liquor.

Results:—

Dry wt:— 75.0 g

Yield (% w/w):— 0.75

Purity:— 99.8%

### Example—3

### Preparation of Memantine hydrochloride

Charge PEG-400 (400 ml) at 25-30° C. Charge N-acetamido-3,5-dimethyl adamantane (100 gm) at 25-30° C. Charge sodium hydroxide (100 gm) at 25-30° C. Raise temperature of reaction mixture to 140° C. Maintain temperature of reaction mixture at 140-145° C. for 12 hours. Cool reaction mixture to 70° C. Add toluene into reaction mixture at 60-70° C. Stir for 15 min. Add DM water and stir for 15 min. Settle the layers. Separate aqueous layer. Add toluene into aqueous layer at 40-60° C. Stir for 15 min and settle the layers. Separate aqueous layer. Combine organic layers of step. Add DM water into toluene layer. Stir for 15 min. Settle the layer at and separate aqueous layer. Add DM water into toluene layer. Stir for 15 min. Settle the layer at and separate aqueous layer. Add charcoal (5 gm) to toluene layer. Stir for 30 min. Filter the solution through hyflo. Wash hyflo bed with toluene. Add 30% conc. HCl (65 gm) to reaction mass at 25-30° C. in 30 min. Stir reaction mass for 30 min and filter solid under vacuum. Wash solid with toluene. Dry solid at 55-60° C. under vacuum. 2^{nd} crop is isolated form mother liquor

Results:—

Dry wt:— 85.0 g

Yield (% w/w):— 0.85

Purity:— 99.5%

### Example—4

### Purification of Crude Memantine hydrochloride

Charge IPA (1500 ml) in to the rbf at 25-30° C. Add crude Memantine HC1 (100 g) to the rbf at 25-30 ° C. Start stirring and raise the temperature up to reflux. Stir it for 15 min at reflux temp. Allow reaction mass to come to 65-70° C. Add charcoal (5 g) to the reaction mass at 65-70° C. Stir it for 15-30 min at 65-70° C. Filter it through hyflo and wash it with hot (65-70° C.) IPA (100 ml). Distill out IPA atmospherically till the volume of reaction mass remains 4 times volume the crude Memantine HCl. Allow reaction mass to come to 25-30° C. Chill it to 0-5° C. Stir it for 1 hr at 0-5° C. Filter it and wash it with chilled (0-5° C.) IPA (50 ml). Suck dry the solid. Dry the solid under vacuum at 60° C. for 12-15 hr. 2^{nd} crop is isolated from the mother liquor.

Results:—

Dry wt:— 80.0 g

Purity:— 99.9%

### Example—5

### Purification of Crude Memantine hydrochloride

Charge mixture of Methanol: Acetone (1000 ml, 1:1) in to the rbf. Add Memantine HCl crude (100 g) to the rbf at 25-30° C. Start stirring and raise the temperature up to 55° C. Stir it for 15-30 min at 55° C. Reaction mass allow to come to 45° C. Add charcoal (5 g) to the reaction mass at 45° C. Stir it for 15-30 min at 45° C. Filter it through hyflo bed and wash it with hot mixture of Methanol: Acetone (1:1, 100 ml). Distill out Methanol: Acetone mixture atmospherically up to 65° C. Degas the residue under vacuum for 1 hr at 60° C. Add Acetone (500 ml) to the residue at 50-60° C. Reflux the reaction mass for 30 min. Allow reaction mass to cool to 25-30° C. Stir it for 1 hr at 25-30° C. Filter it and wash it with Acetone (50 ml). Suck dry the solid. Dry the solid under vacuum at 60° C. for 10-12 hr.

Results:—

Dry wt:— 95.0 g

Purity:— 99.9%

1-4. (canceled)

5. An improved process for the preparation of compound of formula (III) comprising of carrying out bromination of compound of formula (IV) using bromine and characterized by carrying out said bromination in the presence of catalytic amount of HBr in acetic acid.

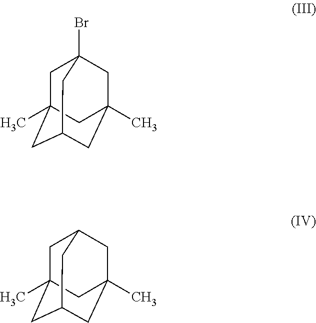

6. An improved process for the preparation of compound of formula (I) comprising steps of:
(a) carrying out bromination of compound of formula (IV) using bromine and characterized by carrying out said bromination in the presence of catalytic amount of HBr in acetic acid to obtain compound of formula (III) 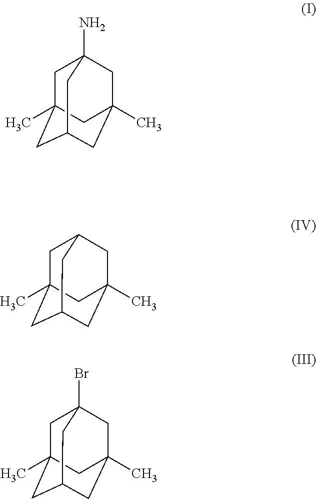
(b) converting compound of formula (III) in the presence of acetonitrile and sulphuric acid to compound of formula (II) 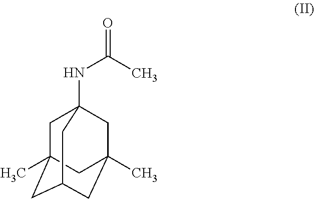
(c) converting compound of formula (II) to Memantine of formula (I) or pharmaceutically acceptable salts thereof.

7. An improved process for the preparation of compound of formula (I) comprising a step of carrying out bromination of compound of formula (IV) using bromine and characterized by carrying out said bromination in the presence of catalytic amount of HBr in acetic acid to obtain compound of formula (III).

8. A process claimed in claim 5 wherein said bromination is carried out using about 4 to 5 molar equivalents of bromine with respect to compound of formula (IV).

9. A process claimed in claim 6 wherein said bromination is carried out using about 4 to 5 molar equivalents of bromine with respect to compound of formula (IV).

10. A process claimed in claim 7 wherein said bromination is carried out using about 4 to 5 molar equivalents of bromine with respect to compound of formula (IV).
